(19) United States
(12) Patent Application Publication
(10) Pub. No.: US 2025/0102465 A1
(43) Pub. Date: Mar. 27, 2025

(54) DISK DEVICE AND METHOD OF INSPECTING DISK DEVICE

(71) Applicants: KABUSHIKI KAISHA TOSHIBA, Tokyo (JP); TOSHIBA ELECTRONIC DEVICES & STORAGE CORPORATION, Tokyo (JP)

(72) Inventor: Akiyo MIZUTANI, Kawasaki Kanagawa (JP)

(21) Appl. No.: 18/589,382

(22) Filed: Feb. 27, 2024

(30) Foreign Application Priority Data
Sep. 21, 2023 (JP) 2023-156366

(51) Int. Cl. G01N 29/02; G01N 29/24; G01N 33/00; G11B 33/06

(52) U.S. Cl. CPC G01N 29/022; G01N 29/2443; G01N 33/0027; G11B 33/06; G01N 2291/021

(57) ABSTRACT

A disk device according to one embodiment includes a housing, a magnetic disk, a circuit board, a first oscillator, and a first adsorption film. The housing is provided with an internal space. The magnetic disk is disposed in the internal space. The circuit board is attached to the housing outside the internal space. The first oscillator is disposed away from an electric circuit in the internal space. The electric circuit is electrically connected to the circuit board. The first adsorption film is formed on the first oscillator, exposed to the internal space, and configured to adsorb a first substance.

### CROSS-REFERENCE TO RELATED APPLICATIONS

This application is based upon and claims the benefit of priority from Japanese Patent Application No. 2023-156366, filed on Sep. 21, 2023; the entire contents of which are incorporated herein by reference.

### FIELD

Embodiments described herein relate generally to a disk device and a method of inspecting a disk device.

### BACKGROUND

A disk device such as a hard disk drive (HDD) typically includes a housing that accommodates various components. The disk device may further include a sensor that detects predetermined substances in the internal space of the housing. The disk device uses such a sensor to be able to detect, for example, entry or occurrence of a predetermined substance in the internal space of the housing.

As an example, a controller of the disk device is electrically connected to the sensor in order to allow the sensor to monitor the entry of the predetermined substance into the internal space and the occurrence of the substance therein. Such a configuration may result in complicating the wiring of an electric circuit to which the controller and the sensor are connected.

### BRIEF DESCRIPTION OF THE DRAWINGS

FIG. 1 is an exemplary plan view illustrating an HDD according to a first embodiment;

FIG. 2 is an exemplary cross-sectional view partially illustrating the HDD of the first embodiment along line F2-F2 in FIG. 1;

FIG. 3 is an exemplary front view illustrating a first gas sensor and a second gas sensor of the first embodiment;

FIG. 4 is an exemplary diagram schematically illustrating the HDD, the first gas sensor, and an inspection device of the first embodiment; and

FIG. 5 is an exemplary plan view illustrating an HDD according to a second embodiment.

### DETAILED DESCRIPTION

A disk device according to one embodiment includes a housing, a magnetic disk, a circuit board, a first oscillator, and a first adsorption film. The housing is provided with an internal space. The magnetic disk is disposed in the internal space. The circuit board is attached to the housing outside the internal space. The first oscillator is disposed away from an electric circuit in the internal space. The electric circuit is electrically connected to the circuit board. The first adsorption film is formed on the first oscillator, exposed to the internal space, and configured to adsorb a first substance.

### First Embodiment

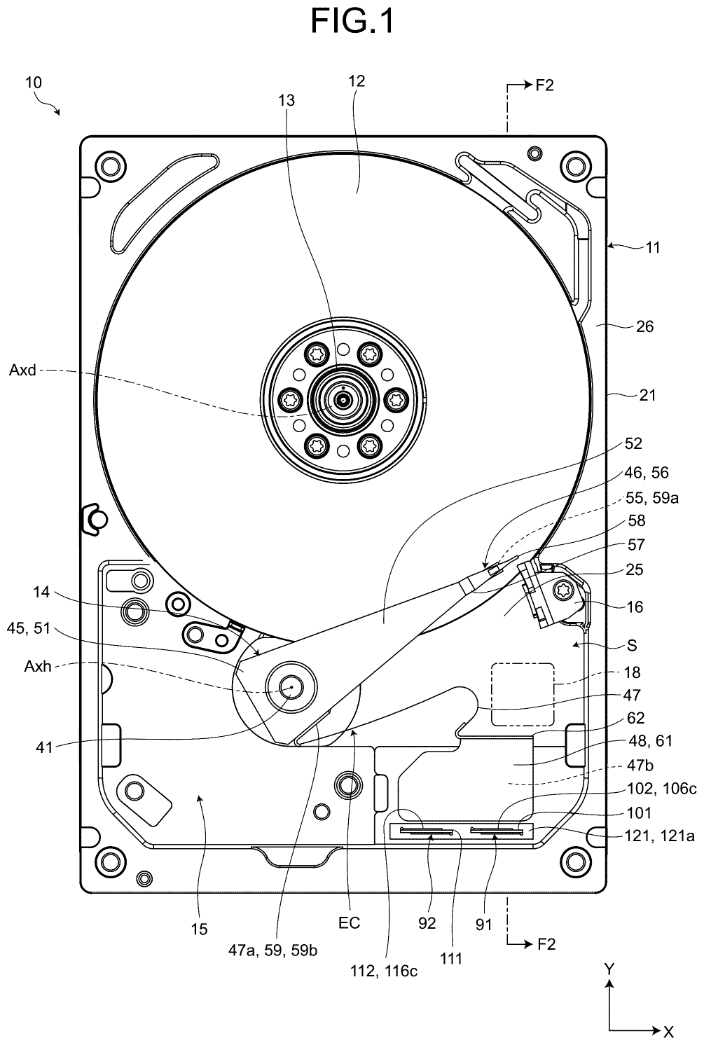

Hereinafter, a first embodiment will be described with reference to FIGS. 1 to 4. Note that in the present specification, components according to embodiments and descriptions of the components may be described in a plurality of expressions. The components and the description thereof are examples, and are not limited by the expressions of the present specification. The components may also be identified with names different from those in the present specification. In addition, the components may be described by expressions different from the expressions of the present specification.

In the following description, “suppress” is defined as, for example, preventing the occurrence of an event, an action, or an influence, or reducing the degree of the event, the action, or the influence. Furthermore, in the following description, “restrict” is defined as, for example, preventing movement or rotation, or allowing movement or rotation within a predetermined range and preventing movement or rotation beyond the predetermined range.

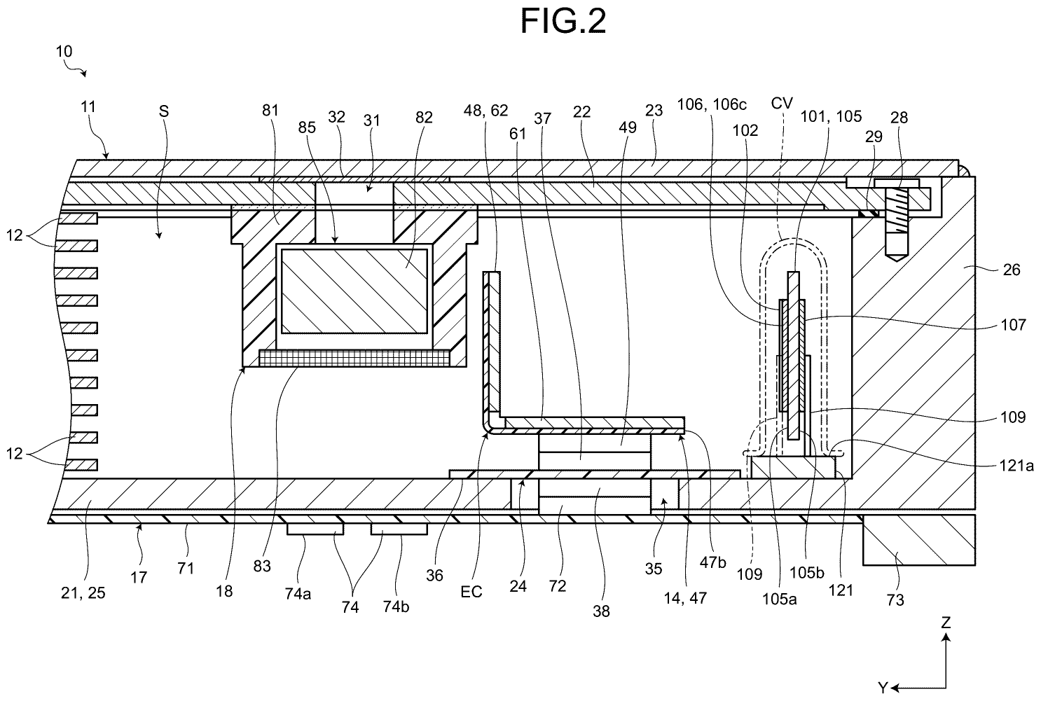

FIG. 1 is an exemplary plan view illustrating a hard disk drive (HDD) 10 according to the first embodiment. FIG. 2 is an exemplary cross-sectional view partially illustrating the HDD 10 of the first embodiment along line F2-F2 in FIG. 1. The HDD 10 is an example of a disk device, and may also be referred to as an electronic device, a storage device, an external storage device, or a magnetic disk device.

As illustrated in the drawings, in the present specification, an X axis, a Y axis, and a Z axis are defined for convenience. The X axis, the Y axis, and the Z axis are orthogonal to each other. The X axis is provided along the width of the HDD 10. The Y axis is provided along the length of the HDD 10. The Z axis is provided along the thickness of the HDD 10.

Furthermore, in the present specification, an X direction, a Y direction, and a Z direction are defined. The X direction is a direction along the X axis, and includes a +X direction indicated by the arrow of the X axis and a −X direction, which is an opposite direction of the arrow of the X axis. The Y direction is a direction along the Y axis, and includes a +Y direction indicated by the arrow of the Y axis and a −Y direction, which is an opposite direction of the arrow of the Y axis. The Z direction is a direction along the Z axis, and includes a +Z direction indicated by the arrow of the Z axis and a-Z direction, which is an opposite direction of the arrow of the Z axis.

The HDD 10 includes a housing 11, a plurality of magnetic disks 12, a spindle motor 13, a head stack assembly (HSA) 14, a voice coil motor (VCM) 15, and a ramp load mechanism 16 illustrated in FIG. 1, and a printed circuit board (PCB) 17 and a breather filter 18 illustrated in FIG. 2. The magnetic disk 12 may also be referred to as a disk or a platter. The breather filter 18 is an example of a filter. The PCB 17 is also referred to as a printed circuit board assembly (PCBA), and is an example of a circuit board.

As illustrated in FIG. 2, the housing 11 includes a base 21, an inner cover 22, an outer cover 23, and a relay board 24. Note that the housing 11 is not limited to this example. Note that in FIG. 1, the inner cover 22 and the outer cover 23 are omitted for the sake of understanding.

As illustrated in FIG. 1, the base 21 has a substantially rectangular parallelepiped box shape opened in the +Z direction. The base 21 has a bottom wall 25 and a side wall 26. The bottom wall 25 has a substantially rectangular (quadrangular) plate shape that expands so as to be substantially orthogonal to the Z direction. The side wall 26 protrudes substantially in the +Z direction from an edge of the bottom wall 25 and has a substantially rectangular frame shape.

The base 21 has an internal space S. The internal space S is formed (defined or sectioned) by the bottom wall 25 and the side wall 26. The side wall 26 surrounds the internal space S. The plurality of magnetic disks 12, the spindle motor 13, the HSA 14, the VCM 15, the ramp load mechanism 16, and the breather filter 18 are disposed in the internal space S.

As illustrated in FIG. 2, the inner cover 22 is attached to the base 21. For example, the inner cover 22 is attached to an end portion of the side wall 26 in the +Z direction by, for example, a screw 28. As a result, the inner cover 22 closes the internal space S.

An endless gasket 29 is interposed between the inner cover 22 and the side wall 26. The gasket 29 hermetically seals a gap between the inner cover 22 and the side wall 26. The gasket 29 is made of, for example, synthetic rubber having low helium permeability. Note that the gasket 29 may be made of another material.

The outer cover 23 covers the inner cover 22 via a gap and is attached to the base 21. The gap between the inner cover 22 and the outer cover 23 may be filled with, for example, a double-sided tape that connects the inner cover 22 and the outer cover 23.

For example, the outer cover 23 is attached to the end portion of the side wall 26 in the +Z direction by welding. As a result, the inner cover 22 and the outer cover 23 hermetically close the internal space S. Note that the outer cover 23 may be omitted.

The inner cover 22 is provided with a vent 31. The vent 31 penetrates the inner cover 22 substantially in the Z direction and communicates with the internal space S. After components are disposed in the internal space S and the inner cover 22 is attached to the base 21, air in the internal space S is removed from the vent 31. Furthermore, the internal space S is filled with a gas different from air through the vent 31.

The gas filling the internal space S is, for example, a low density gas having a density lower than that of air, an inert gas having low reactivity, or the like. For example, the internal space S is filled with helium. Helium is an example of a first gas. Note that the internal space S may be filled with another fluid.

The housing 11 further includes a seal 32. The seal 32 is, for example, an aluminum seal. The seal 32 hermetically closes the vent 31. As a result, the seal 32 restricts leakage of helium from the internal space S through the vent 31 to the outside of the housing 11. Note that the outer cover 23 may be provided with another vent. In this case, the seal 32 closes the vent of the outer cover 23 instead of the vent 31.

The bottom wall 25 is provided with a through hole 35. The through hole 35 penetrates the bottom wall 25 substantially in the Z direction so as to communicate the internal space S with the outside. The relay board 24 is attached to the bottom wall 25 so as to hermetically close the through hole 35. The relay board 24 includes a board 36 and two relay connectors 37 and 38.

The board 36 is, for example, a rigid board such as a glass epoxy board. The board 36 covers the through hole 35 and is attached to the bottom wall 25. The board 36 hermetically closes the through hole 35. The two relay connectors 37 and 38 are mounted on both surfaces of the board 36 and are electrically connected to each other.

As illustrated in FIG. 1, the plurality of magnetic disks 12 extend orthogonally to the Z direction. The plurality of magnetic disks 12 are arranged at intervals in the Z direction. Magnetic recording layers are provided on both surfaces of the magnetic disk 12.

The spindle motor 13 supports the plurality of magnetic disks 12. The plurality of magnetic disks 12 are held to a hub of the spindle motor 13 by, for example, a clamp spring.

The spindle motor 13 integrally rotates the plurality of magnetic disks 12 about an axis Axd. The axis Axd is a virtual axis extending substantially in the Z direction. The axis Axd is, for example, the center of rotation of the magnetic disk 12 and the spindle motor 13, and is also the axis of the magnetic disk 12.

The housing 11 further includes a support shaft 41 separated from the magnetic disk 12. The support shaft 41 protrudes, for example, from the bottom wall 25 substantially in the +Z direction. The HSA 14 is rotatably supported by the support shaft 41.

The HSA 14 can rotate about an axis Axh. The axis Axh is a virtual axis extending substantially in the Z direction. The axis Axh is, for example, the center of rotation of the HSA 14 and the axis of the support shaft 41.

The HSA 14 includes a carriage 45, a plurality of head gimbal assemblies (HGAs) 46, a flexible printed circuit board (FPC) 47, a support plate 48, and a relay connector 49 in FIG. 2. As illustrated in FIG. 1, the carriage 45 includes an actuator block 51 and a plurality of arms 52.

The actuator block 51 is attached to the support shaft 41 via a bearing so as to be rotatable about the axis Axh, for example. The plurality of arms 52 protrude from the actuator block 51 substantially in parallel in a direction substantially orthogonal to the axis Axh.

Each of the plurality of HGAs 46 includes a magnetic head 55 and a suspension 56. The magnetic head 55 may also be referred to as a slider. The magnetic head 55 records and reproduces information on and from a corresponding one of the plurality of magnetic disks 12. In other words, the magnetic head 55 reads and writes information from and to the magnetic disk 12. The suspension 56 is attached to the arm 52 and holds the magnetic head 55.

The suspension 56 includes a base plate 57, a load beam 58, and a flexure 59. The base plate 57 is attached to a distal end of the arm 52. The load beam 58 has a plate shape thinner than the base plate 57. The load beam 58 is attached to the base plate 57 so as to extend from the base plate 57.

The flexure 59 is a kind of flexible printed wiring board of an elongated belt shape. The flexure 59 includes, for example, a metallic backing plate, an insulating base layer, a conductive layer, and an insulating cover layer.

The flexure 59 extends along the arm 52, the base plate 57, and the load beam 58. A rotatable gimbal is provided at one end portion 59a of the flexure 59. The magnetic head 55 is attached to the gimbal of the flexure 59.

One end portion 47a of the FPC 47 is attached to the actuator block 51. The other end portions 59b of the plurality of flexures 59 are connected to the end portion 47a of the FPC 47. Therefore, the flexure 59 electrically connects the magnetic head 55 and the FPC 47.

The other end portion 47b of the FPC 47 is attached to the support plate 48. The support plate 48 is made of, for example, metal or synthetic resin. The rigidity of the support plate 48 is higher than the rigidity of the FPC 47. The support plate 48 includes an attachment part 61 and an upright part 62.

As illustrated in FIG. 2, the attachment part 61 is disposed along the bottom wall 25 of the base 21, and is attached to the bottom wall 25 by, for example, a screw. The end portion 47b of the FPC 47 is attached to the attachment part 61.

The relay connector 49 is mounted on the end portion 47b. The attachment part 61 reinforces the end portion 47b of the FPC 47 on which the relay connector 49 is mounted. The relay connector 49 is connected to the relay connector 37 of the relay board 24.

The upright part 62 extends substantially in the Z direction from the attachment part 61. The upright part 62 supports the FPC 47 such that the width of the FPC 47 substantially coincides with the Z direction between the two end portions 47a and 47b. The upright part 62 is disposed, for example, along an X-Z plane. Note that the upright part 62 is not limited to this example.

The VCM 15 in FIG. 1 includes a voice coil, a pair of yokes, and a magnet provided on the yoke. The voice coil is held by the actuator block 51. The VCM 15 moves the magnetic head 55 to a desired position by rotating the carriage 45 around the axis Axh.

When the magnetic head 55 moves to an outer edge of the magnetic disk 12 due to the rotation of the HSA 14 by the VCM 15, the ramp load mechanism 16 holds the magnetic head 55 at a position separated from the magnetic disk 12.

As illustrated in FIG. 2, the PCB 17 is disposed outside the internal space S, and is attached to the bottom wall 25 with, for example, a screw. In other words, the PCB 17 is attached to the housing 11 outside the internal space S.

The PCB 17 includes a printed wiring board (PWB) 71, a relay connector 72, an interface (I/F) connector 73, and a plurality of electronic components 74. The PCB 17 may further include other components.

The PWB 71 is, for example, a rigid board such as a glass epoxy board, and is a multilayer board, a build-up board, or the like. The relay connector 72, the I/F connector 73, and the plurality of electronic components 74 are mounted on the PWB 71.

The relay connector 72 is connected to the relay connector 38 of the relay board 24. As a result, the PCB 17 is electrically connected to the VCM 15 through the relay board 24 and the FPC 47, and is electrically connected to the magnetic head 55 through the relay board 24, the FPC 47, and the flexure 59. Furthermore, the PCB 17 is electrically connected to the spindle motor 13 through, for example, another FPC. That is, an electric circuit EC electrically connected to the PCB 17 is disposed in the internal space S.

The electric circuit EC is one component or a set of components electrically connected to the PCB 17 and forming at least one electric circuit together with at least a part of the PCB 17. The electric circuit EC includes, for example, the spindle motor 13, the VCM 15, the relay board 24, the FPC 47, the magnetic head 55, and the flexure 59. Note that the electric circuit EC is not limited to this example.

The I/F connector 73 is, for example, a serial ATA (SATA) connector, and is connected to a host computer. The HDD 10 is supplied with power from the host computer through the I/F connector 73 and communicates with the host computer.

The plurality of electronic components 74 include, for example, a processor 74a, a memory 74b such as a ROM, a RAM, and a flash memory, and various components. The processor 74a may also be referred to as a controller.

The processor 74a controls the entire HDD 10 based on, for example, a program read from the memory 74b. For example, the processor 74a controls the spindle motor 13 through the PWB 71. Furthermore, the processor 74a controls the VCM 15 and the magnetic head 55 through the PWB 71, the relay board 24, the FPC 47, and the flexure 59. For example, the processor 74a may acquire information from various sensors disposed inside the housing 11.

The processor 74a may control the entire HDD 10 in cooperation with wiring of the PWB 71 and the other electronic components 74. That is, the entire PCB 17 including the PWB 71 and the plurality of electronic components 74 can be a control device of the HDD 10.

The breather filter 18 includes, for example, a case 81, an adsorbent 82, and a membrane filter 83. The case 81 is made of, for example, synthetic resin having low helium permeability or metal. The case 81 is attached to the inner cover 22 so as to cover the vent 31. The case 81 has a chamber 85 communicating with the vent 31 and the internal space S inside.

The adsorbent 82 is disposed in the chamber 85. The adsorbent 82 is, for example, a gas collector such as activated carbon, and adsorbs various organic substances including a gas. The adsorbent 82 may adsorb other substances. The membrane filter 83 is attached to the case 81 so as to cover the chamber 85.

As illustrated in FIG. 1, the HDD 10 further includes a first gas sensor 91 and a second gas sensor 92. Each of the first gas sensor 91 and the second gas sensor 92 is a quartz crystal microbalance (QCM) sensor. The first gas sensor 91 and the second gas sensor 92 are disposed in the internal space S.

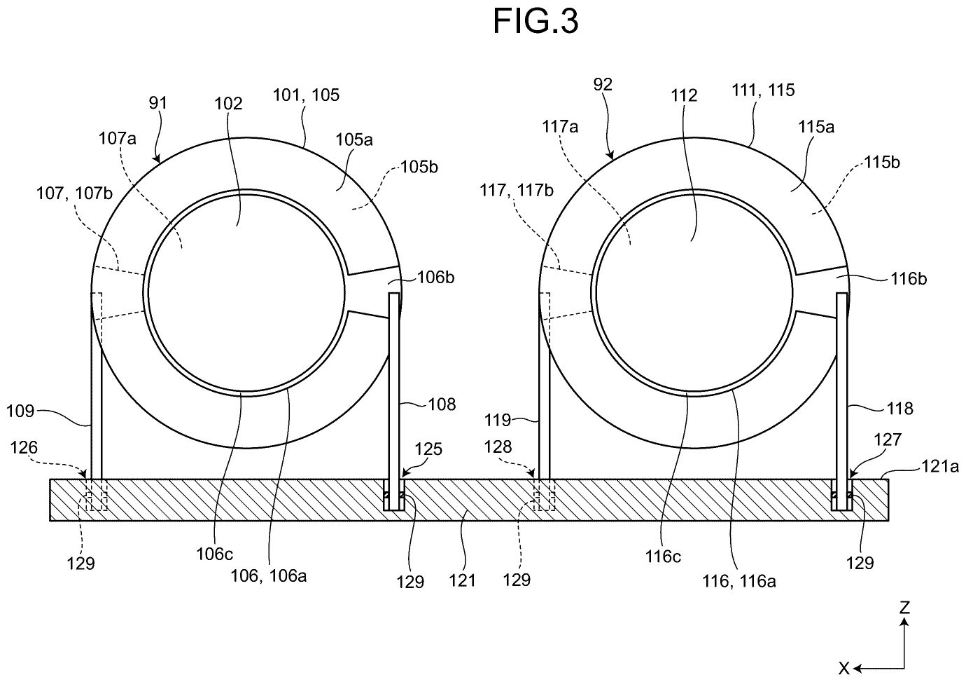

FIG. 3 is an exemplary front view illustrating the first gas sensor 91 and the second gas sensor 92 of the first embodiment. The first gas sensor 91 includes an oscillator 101 and an adsorption film 102. The oscillator 101 is an example of a first oscillator. The adsorption film 102 is an example of a first adsorption film.

The oscillator 101 is, for example, a crystal oscillator. Note that the oscillator 101 may be another oscillator or resonator such as a ceramic resonator.

The oscillator 101 includes a piezoelectric body 105, two electrodes 106 and 107, and two lead wires 108 and 109. The electrode 106 is an example of a first electrode. The electrode 107 is an example of a second electrode. The lead wire 108 is an example of a first lead. The lead wire 109 is an example of a second lead.

The piezoelectric body 105 is, for example, crystal. The piezoelectric body 105 has a substantially disk shape. The piezoelectric body 105 has two surfaces 105a and 105b. The surface 105a is an example of a first surface. The surface 105b is an example of a second surface.

Each of the surfaces 105a and 105b has a substantially circular shape. The surface 105a faces the plurality of magnetic disks 12, the FPC 47, the plurality of arms 52, and the plurality of HGAs 46. The surface 105b is opposite to the surface 105a.

The electrode 106 is a metal film formed on the surface 105a. The electrode 106 has a circular part 106a and a connecting part 106b. The circular part 106a is concentric with the surface 105a and is smaller in size than the surface 105a. The connecting part 106b protrudes from an edge of the circular part 106a along the surface 105a.

The circular part 106a has an outer surface 106c. The outer surface 106c has a substantially circular shape. As illustrated in FIG. 1, the outer surface 106c faces the plurality of magnetic disks 12, the FPC 47, the plurality of arms 52, and the plurality of HGAs 46.

The electrode 107 in FIG. 3 is a metal film formed on the surface 105b. The electrode 107 includes a circular part 107a and a connecting part 107b. The circular part 107a is concentric with the surface 105b and smaller in size than the surface 105b. The piezoelectric body 105 is located between the circular part 106a of the electrode 106 and the circular part 107a of the electrode 107. The connecting part 107b protrudes from an edge of the circular part 107a along the surface 105b. A direction in which the connecting part 106b protrudes and a direction in which the connecting part 107b protrudes are different from each other.

The lead wire 108 is connected to the connecting part 106b of the electrode 106. The lead wire 109 is connected to the connecting part 107b of the electrode 107. The lead wires 108 and 109 extend substantially in parallel. Note that the lead wires 108 and 109 may extend in different directions or may be bent.

The adsorption film 102 is formed on the outer surface 106c of the electrode 106. Note that the adsorption film 102 may be formed on another part of the oscillator 101. The adsorption film 102 adsorbs various organic substances including gas and is exemplified by a carbon film.

In the present embodiment, the adsorption film 102 adsorbs outgas. The outgas is an example of a first substance and a second gas. The outgas is a gas different from helium filling the internal space S.

The outgas is, for example, a volatile organic compound (VOC). In the following description, the outgas includes not only a VOC released from the components of the HDD 10 but also a VOC entering the internal space S from the outside. In other words, the outgas refers to gases adsorbed by the adsorption film 102 in the following.

The adsorption film 102 is exposed in the internal space S. In other words, the adsorption film 102 is exposed to a gas or gases present in the internal space S, such as helium. Note that the adsorption film 102 may be partially covered.

The second gas sensor 92 has substantially the same configuration as the first gas sensor 91. The second gas sensor 92 includes an oscillator 111 and an adsorption film 112. The oscillator 111 is an example of a second oscillator. The adsorption film 112 is an example of a second adsorption film.

The oscillator 111 is a crystal oscillator as with the oscillator 101 of the first gas sensor 91. The oscillator 111 includes a piezoelectric body 115, two electrodes 116 and 117, and two lead wires 118 and 119.

The piezoelectric body 115 has a substantially disk shape. The piezoelectric body 115 has two surfaces 115a and 115b. Each of the surfaces 115a and 115b has a substantially circular shape. The surface 115a faces the plurality of magnetic disks 12, the FPC 47, the plurality of arms 52, and the plurality of HGAs 46. The surface 115b is opposite to the surface 115a.

The electrode 116 is a metal film provided on the surface 115a. The electrode 116 has a circular part 116a and a connecting part 116b. The circular part 116a is concentric with the surface 115a and is smaller in size than the surface 115a. The connecting part 116b protrudes from an edge of the circular part 116a along the surface 115a.

The circular part 116a has an outer surface 116c. The outer surface 116c has a substantially circular shape. As illustrated in FIG. 1, the outer surface 116c faces the plurality of magnetic disks 12, the FPC 47, the plurality of arms 52, and the plurality of HGAs 46.

The electrode 117 in FIG. 3 is a metal film formed on the surface 115b. The electrode 117 has a circular part 117a and a connecting part 117b. The circular part 117a is concentric with the surface 115b and is smaller than the surface 115b. The connecting part 117b protrudes from an edge of the circular part 117a along the surface 115b. A direction in which the connecting part 116b protrudes and a direction in which the connecting part 117b protrudes are different from each other.

The lead wire 118 is connected to the connecting part 116b of the electrode 116. The lead wire 119 is connected to the connecting part 117b of the electrode 117. The lead wires 118 and 119 extend substantially in parallel.

The adsorption film 112 is formed on the outer surface 116c of the electrode 116. The adsorption film 112 is exposed in the internal space S. The adsorption film 112 is, for example, made of silicon dioxide or Y-type zeolite, and adsorbs siloxane. That is, the adsorption film 112 adsorbs a substance or substances different from the outgas adsorbed by the adsorption film 102. Siloxane is an example of a second substance.

The adsorption films 102 and 112 are not limited to the above examples. For example, the adsorption film 102 or the adsorption film 112 may be a silver film that adsorbs an inorganic gas such as sulfur dioxide or chlorine, or may be another adsorption film. In addition, the adsorption films 102 and 112 may be capable of adsorbing not only gas but also solid or liquid.

The housing 11 further includes a base 121. The base 121 is an example of a holder. The base 121 is disposed in the internal space S and is attached to the bottom wall 25. Note that the base 121 may be integrated with the base 21.

The base 121 has an upper surface 121a. For example, the upper surface 121a is substantially flat, and faces substantially in the +Z direction. The base 121 is provided with a plurality of holes 125, 126, 127, and 128 opened in the upper surface 121a.

The lead wire 108 of the first gas sensor 91 is inserted into the hole 125, and the lead wire 109 is inserted into the hole 126. Furthermore, the lead wire 118 of the second gas sensor 92 is inserted into the hole 127, and the lead wire 119 is inserted into the hole 128.

An O-ring 129 is attached to each of the lead wires 108, 109, 118, and 119. The O-rings 129 fill the gaps between the base 121 and the lead wires 108, 109, 118, and 119 inside the holes 125, 126, 127, and 128. As a result, the base 121 detachably holds the lead wires 108, 109, 118, and 119 via the O-rings 129.

The lead wires 108, 109, 118, and 119 may be held in the housing 11 by another method. For example, the lead wires 108, 109, 118, and 119 may be held by a clip or may be attached to the housing 11 with an adhesive.

As illustrated in FIG. 2, in the present embodiment, the base 121 is located between the relay board 24 and the side wall 26. The base 121, the first gas sensor 91, and the second gas sensor 92 are farther separated from the magnetic disk 12 than the breather filter 18, the relay board 24, and the FPC 47. Note that the positions of the base 121, the first gas sensor 91, and the second gas sensor 92 are not limited to this example.

The base 121 is made of an insulating material or has a surface covered with an insulating material. In addition, the base 121 is not electrically connected to the electric circuit EC. Thus, the lead wires 108, 109, 118, and 119 are not electrically connected to the electric circuit EC. Furthermore, the oscillators 101 and 111 are separated from the components other than the base 121 and the O-ring 129. The oscillators 101 and 111 are thus separated from the electric circuit EC. In other words, the oscillators 101 and 111 are electrically disconnected from the electric circuit EC. Note that the oscillators 101 and 111 and the electric circuit EC may be connected to a common ground.

For example, the oscillators 101 and 111 may be mounted on a board independent of the electric circuit EC. In this case, the board may be connected to the oscillators 101 and 111 so as to be energizable. However, the board is electrically disconnected from the electric circuit EC and is supplied with no power from the host computer via the I/F connector 73.

Since the oscillators 101 and 111 are separated from the electric circuit EC, the PCB 17 does not cause the oscillators 101 and 111 to oscillate during operation of the HDD 10. However, even when the oscillators 101 and 111 do not oscillate, the adsorption film 102 adsorbs outgas in the internal space S, and the adsorption film 112 adsorbs siloxane in the internal space S.

Hereinafter, a part of a method of manufacturing the HDD 10 will be exemplified. Note that the method of manufacturing the HDD 10 is not limited to the following method, and other methods may be used. First, an initial value of an oscillation frequency of the oscillators 101 and 111 is recorded in, for example, the memory 74b. Note that the initial value may be recorded in a storage area of the magnetic disk 12.

As described above, the memory 74b or the storage area of the magnetic disk 12 stores the initial values of the oscillation frequency of the oscillators 101 and 111. The memory 74b or the storage area of the magnetic disk 12 is an example of a storage.

The initial values of the oscillation frequency of the oscillators 101 and 111 is, for example, the oscillation frequencies (resonance frequencies) of the oscillators 101 and 111 measured before the manufacturing process. Note that the initial value may be provided from the manufacturer of the oscillators 101 and 111.

Next, the plurality of magnetic disks 12, the spindle motor 13, the HSA 14, the VCM 15, and the ramp load mechanism 16 are accommodated in the internal space S. The magnetic disk 12, the spindle motor 13, the HSA 14, the VCM 15, and the ramp load mechanism 16 are operably assembled as the HDD 10.

Next, the lead wires 108, 109, 118, and 119 of the first gas sensor 91 and the second gas sensor 92 are inserted into the holes 125, 126, 127, and 128. As a result, the lead wires 108, 109, 118, and 119 are held on the base 121.

When the first gas sensor 91 and the second gas sensor 92 are disposed in the internal space S, the first gas sensor 91 and the second gas sensor 92 are temporarily covered with a cover CV virtually indicated by a two-dot chain line in FIG. 2, for example. The cover CV is made of, for example, an insulating material such as synthetic rubber.

The cover CV covers the adsorption films 102 and 112. As a result, the cover CV can suppress contamination of the adsorption films 102 and 112 in the manufacturing process of the HDD 10. An operator or a mounter grips the first gas sensor 91 or the second gas sensor 92 via the cover CV. When the first gas sensor 91 and the second gas sensor 92 are disposed in the internal space S, the cover CV is removed.

Next, the inner cover 22 is attached to the base 21 by, for example, the screw 28. Next, air in the internal space S is removed through the membrane filter 83, the adsorbent 82, and the vent 31. Furthermore, the internal space S is filled with helium through the vent 31, the adsorbent 82, and the membrane filter 83. Note that air may be discharged from the internal space S by filling the internal space S with helium.

The adsorbent 82 adsorbs outgas from helium while flowing into the internal space S through the vent 31, if any outgas is contained therein. The membrane filter 83 may collect outgas. In this manner, the breather filter 18 collects outgas from gas flow passing through the vent 31. The gas flow includes airflow.

Next, the seal 32 is attached to the inner cover 22. As a result, the vent 31 is sealed by the seal 32. The vent 31 is hermetically sealed by the seal 32. Furthermore, the gap between the base 21 and the inner cover 22 is hermetically sealed by the gasket 29. As a result, leakage of helium from the internal space S is prevented at least temporarily.

Next, the outer cover 23 is joined to the side wall 26 of the base 21 by, for example, welding. As a result, the outer cover 23 covering the inner cover 22 is fixed to the base 21. Thus, the manufacture of the HDD 10 is completed. Note that before the outer cover 23 is attached to the base 21, the operation of the HDD 10 and the leakage of helium may be inspected.

In the internal space S of the HDD 10 described above, the rotation of the magnetic disk 12 and the HSA 14 causes gas flow. The adsorption films 102 and 112 of the first gas sensor 91 and the second gas sensor 92 are exposed to the gas flow. The adsorption films 102 and 112 adsorb a predetermined substance from the gas flow.

When a failure occurs in the HDD 10, analysis of the cause may be performed. For example, outgas released from the components in the internal space S or ambient air entering the internal space S from the outside may cause a failure in the HDD 10.

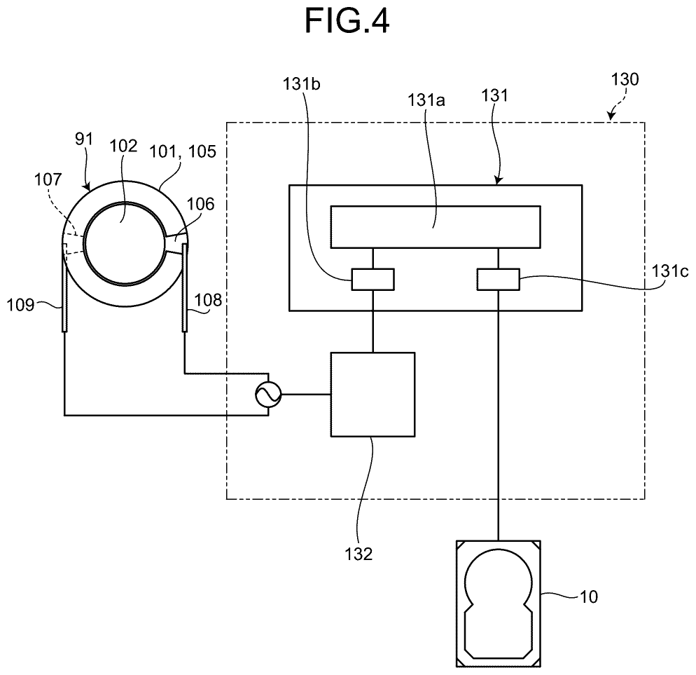

FIG. 4 is an exemplary diagram schematically illustrating the HDD 10, the first gas sensor 91, and an inspection device 130 of the first embodiment. The inspection device 130 can analyze the cause of a failure of the HDD 10. As illustrated in FIG. 4, the inspection device 130 includes, for example, an information processing device 131 and a frequency meter 132. Note that the inspection device 130 is not limited to this example.

The information processing device 131 is, for example, a general computer. The information processing device 131 includes a control device 131a such as a CPU, a storage device such as a ROM or a RAM, an external storage device such as an HDD or a CD drive device, a display device such as a display, and an input device such as a keyboard or a mouse.

The control device 131a is electrically connected to the frequency meter 132 through an interface (I/F) 131b. Furthermore, the control device 131a is electrically connected to the HDD 10 to be an inspected through an I/F 131c.

Hereinafter, a part of a method of inspecting the HDD 10 will be exemplified. Note that the method of inspecting the HDD 10 is not limited to the following method, and other methods may be used. First, the control device 131a acquires the initial value of the oscillation frequency of the oscillator 101 from the memory 74b of the HDD 10.

The memory 74b stores not only the initial value of the oscillation frequency of the oscillator 101 but also various types of information. The control device 131a further acquires, for example, an operating time of the HDD 10 and a temperature transition of the internal space S from the memory 74b.

Next, the first gas sensor 91 and the second gas sensor 92 are taken out from the internal space S of the HDD 10. For example, the outer cover 23 is opened, the inner cover 22 is detached from the base 21, and the lead wires 108, 109, 118, and 119 are removed from the holes 125, 126, 127, and 128.

Next, the frequency meter 132 applies a voltage to the oscillator 101 of the first gas sensor 91. Specifically, the frequency meter 132 causes an alternating current to flow through the lead wires 108 and 109 of the oscillator 101. As a result, the oscillator 101 vibrates.

The frequency meter 132 measures the oscillation frequency (resonance frequency) of the oscillator 101. The frequency meter 132 is, for example, a network analyzer or a frequency counter. The control device 131a acquires the measurement of the oscillation frequency of the oscillator 101 from the frequency meter 132.

Next, the control device 131a calculates a difference between a measured value of the oscillation frequency of the oscillator 101 and the initial value. Next, the control device 131a determines whether the internal space S contained outgas, based on the difference.

The oscillation frequency (resonance frequency) of the oscillator 101 varies according to the mass of substances adsorbed by the adsorption film 102. Thus, the adsorption film 102 adsorbs the outgas contaminating the internal space S, if found, so that the oscillation frequency of the oscillator 101 varies from the initial value.

The control device 131a determines whether or not the difference between the measured value and the initial value is larger than a predetermined threshold. Note that an insignificantly small amount of outgas may accumulate in the adsorption film 102 according to the operating time. In consideration of the accumulation, the threshold is set to increase according to the operating time. The control device 131a sets the threshold based on the operating time of the HDD 10 read from the memory 74b.

When the difference between the measurement value and the initial value is larger than the threshold, the control device 131a determines that outgas existed in the internal space S and the internal space S was contaminated with the outgas. In other words, the inspection device 130 determines that contamination due to outgas is the cause of the failure of the HDD 10.

On the other hand, when the difference is smaller than the threshold, the control device 131a determines that the internal space S was not contaminated with outgas. In other words, the inspection device 130 determines that outgas is not the cause of the failure of the HDD 10.

When it is determined that the internal space S is contaminated with outgas, for example, the outgas adsorbed by the adsorption film 102 is measured by a gas chromatography mass spectrometry (GC-MS) or a time-of-flight secondary ion mass spectrometry (TOF-SIMS). As a result, the component of the outgas can be identified.

When it is determined that there is no contamination from outgassing found in the internal space S, the first gas sensor 91 is detached from the frequency meter 132, and the second gas sensor 92 is connected to the frequency meter 132. The frequency meter 132 applies a voltage to the oscillator 111 of the second gas sensor 92 to vibrate the oscillator 111. The frequency meter 132 measures the oscillation frequency of the oscillator 111. The control device 131a acquires the measurement of the oscillation frequency of the oscillator 111 from the frequency meter 132.

Next, the control device 131a calculates a difference between a measured value of the oscillation frequency of the oscillator 111 and the initial value. Next, the control device 131a determines whether the internal space S contained siloxane, based on the difference. The adsorption film 112 adsorbs the siloxane contaminating the internal space S, if found, causing the oscillation frequency of the oscillator 111 to vary from the initial value.

When the difference between the measurement value and the initial value is larger than the threshold, the control device 131a determines that siloxane exists in the internal space S and the internal space S is contaminated with the siloxane. On the other hand, when the difference is smaller than the threshold, the control device 131a determines that the internal space S is not contaminated with siloxane.

As described above, the inspection device 130 of the present embodiment determines whether the internal space S was contaminated with outgas or siloxane. As a result, the inspection device 130 can investigate whether the failure in the HDD 10 is caused by the contamination in the internal space S.

In the HDD 10 according to the first embodiment described above, the housing 11 is provided with the internal space S. The magnetic disk 12 is disposed in the internal space S. The PCB 17 is attached to the housing 11 outside the internal space S. In the internal space S the oscillator 101 is disposed away from the electric circuit EC which is electrically connected to the PCB 17. The adsorption film 102 formed on the oscillator 101 is exposed in the internal space S, to adsorb outgas therein.

The oscillation frequency of the oscillator 101 varies according to the mass of the outgas adsorbed by the adsorption film 102. This allows inspection of the internal space S for contamination from outgassing based on the oscillation frequency of the oscillator 101 in the HDD 10 of the present embodiment. The oscillator 101 is disposed away from the electric circuit EC, so that the HDD 10 of the present embodiment can avoid complication of the wiring of the electric circuit EC (for example, the FPC 47). Note that the oscillator 101 can be extracted from the internal space S and attached to the inspection device 130 for easier inspection.

The internal space S is filled with helium, which is a gas different from air. The HDD 10 of the present embodiment can allow inspection of the internal space S filled with a gas such as helium for contamination from outgassing.

The adsorption film 102 adsorbs outgas that is a gas different from helium. According to the HDD 10 of the present embodiment, it is possible to examine whether the internal space S filled with a gas such as helium was contaminated with outgas being another gas.

The oscillator 101 has the outer surface 106c facing the magnetic disk 12. The adsorption film 102 is formed on the outer surface 106c and thus exposed to the gas flow generated by the rotation of the magnetic disk 12. The outgas contained in the gas flow, if any, collides with the adsorption film 102. In this manner, the adsorption film 102 can efficiently adsorb the outgas in the internal space S.

The breather filter 18 is disposed in the internal space S. The housing 11 is provided with the vent 31 communicating with the internal space S. The breather filter 18 collects the outgas from the gas flow while passing through the vent 31. For example, while the internal space S becomes filled with helium through the vent 31, outgas is collected from the helium, if any. However, there may be a situation that outgas flows into the internal space S from gaps other than the vent 31 or outgas may occur in the internal space S. In this regard, according to the HDD 10 of the present embodiment the adsorption film 102 can adsorb such outgas, thereby enabling inspection of the internal space S for contamination from outgassing.

The oscillator 101 includes the piezoelectric body 105, the electrodes 106 and 107, and the lead wires 108 and 109. The electrode 106 is disposed on the surface 105a of the piezoelectric body 105. The electrode 107 is disposed on the surface 105b of the piezoelectric body 105, the surface 105b opposite the surface 105a. The lead wire 108 is connected to the electrode 106. The lead wire 109 is connected to the electrode 107. The housing 11 includes the base 121 holding the lead wires 108 and 109. As a result, the HDD 10 of the present embodiment can avoid or reduce the possibility that the piezoelectric body 105 suffer damage, as compared with the piezoelectric body 105 being held.

The base 121 detachably holds the lead wires 108 and 109. This makes it possible to easily extract the oscillator 101 from the internal space S for inspection by, for example, the inspection device 130 located outside.

In the internal space S the oscillator 111 is disposed away from the electric circuit EC which is electrically connected to the PCB 17. The adsorption film 112 formed on the oscillator 111 is exposed in the internal space S. The adsorption film 112 is configured to adsorb siloxane different from outgas. As a result, the HDD 10 of the present embodiment can allow inspection of the internal space S for contamination from a plurality of substances.

The memory 74b stores the initial value of the oscillation frequency of the oscillator 101. This allows comparison between the initial value of the oscillation frequency of the oscillator 101 pre-stored in the memory 74b and the measurement of the oscillation frequency of the oscillator 101 at the time of inspection, to be thereby able to easily examine whether there was contamination from outgassing in the internal space S. In addition, the individual HDDs 10 include the respective memories 74b storing the initial values of the oscillation frequency of their corresponding oscillators 101, which enables the inspection of the internal space S for contamination from outgassing regardless of individual differences among the HDDs 10.

The inspection device 130 applies a voltage to the oscillator 101 of the HDD 10. Then, the inspection device 130 measures the oscillation frequency of the oscillator 101. The inspection device 130 acquires the initial value of the oscillation frequency from the memory 74b. The inspection device 130 calculates the difference between the measured value of the oscillation frequency of the oscillator 101 and the initial value. The inspection device 130 determines whether the internal space S contained outgas, based on the difference. In this manner, the inspection device 130 can easily examine whether the internal space S was contaminated with the outgas.

### Second Embodiment

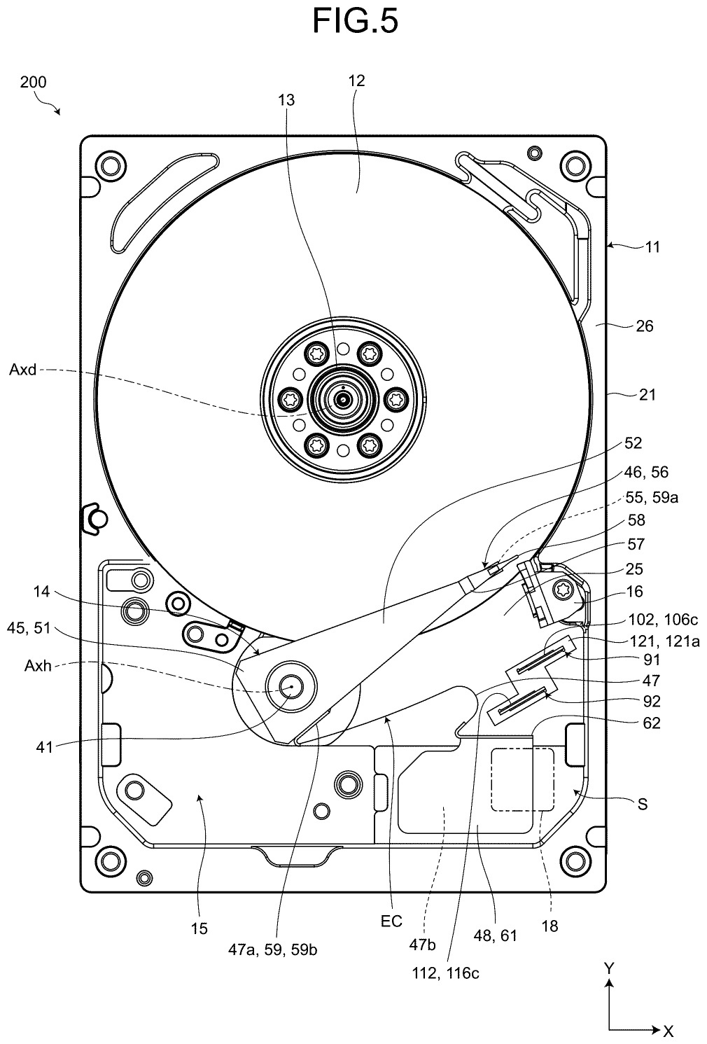

Hereinafter, a second embodiment will be described with reference to FIG. 5. Note that in the following description of the embodiment, components having functions similar to those of the components already described are denoted by the same reference numerals as those of the components already described, and the description thereof may be omitted. In addition, the plurality of components denoted by the same reference numerals do not necessarily have all the functions and properties in common, and may have different functions and properties according to each embodiment.

FIG. 5 is an exemplary plan view illustrating an HDD 200 according to the second embodiment. The HDD 200 of the second embodiment is substantially equal to the HDD 10 of the first embodiment except for the points described below.

In the HDD 200, the base 121, the first gas sensor 91, and the second gas sensor 92 are located between the magnetic disk 12 and the FPC 47. On the other hand, the breather filter 18 is farther separated from the magnetic disk 12 than the upright part 62 of the support plate 48.

In both the first embodiment and the second embodiment, the first gas sensor 91 and the second gas sensor 92 are farther separated from the magnetic disk 12 than the ramp load mechanism 16. Because of this, the first gas sensor 91 and the second gas sensor 92 are less likely to interfere with the HSA 14 being in rotation.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A disk device comprising:
a housing with an internal space;
a magnetic disk disposed in the internal space;
a circuit board attached to the housing outside the internal space;
a first oscillator disposed away from an electric circuit in the internal space, the electric circuit being electrically connected to the circuit board; and
a first adsorption film formed on the first oscillator, exposed to the internal space, and configured to adsorb a first substance.

2. The disk device according to claim 1, wherein
the internal space is filled with a first gas different from air.

3. The disk device according to claim 2, wherein
the first substance includes a second gas different from the first gas.

4. The disk device according to claim 3, wherein
the first oscillator has an outer surface facing the magnetic disk, and
the first adsorption film is formed on the outer surface.

5. The disk device according to claim 2, further comprising
a filter disposed in the internal space, wherein
the housing is provided with a vent communicating with the internal space, and
the filter collects the first substance from gas flow while passing through the vent.

6. The disk device according to claim 1, wherein
the first oscillator includes
a piezoelectric body,
a first electrode disposed on a first surface of the piezoelectric body,
a second electrode disposed on a second surface of the piezoelectric body, the second surface being opposite the first surface,
a first lead connected to the first electrode, and
a second lead connected to the second electrode, and
the housing includes a holder that holds the first lead and the second lead.

7. The disk device according to claim 6, wherein
the holder detachably holds the first lead and the second lead.

8. The disk device according to claim 1, further comprising:
a second oscillator disposed away from the electric circuit; and
a second adsorption film formed on the second oscillator, exposed to the internal space, and configured to adsorb a second substance different from the first substance.

9. The disk device according to claim 1, further comprising:
a storage that stores an initial value of an oscillation frequency of the first oscillator.

10. A method of inspecting the disk device according to claim 9, comprising:
applying, by an inspection device, a voltage to the first oscillator of the disk device;
measuring, by the inspection device, an oscillation frequency of the first oscillator;
acquiring, by the inspection device, the initial value from the storage;
calculating, by the inspection device, a difference between a measured value of the oscillation frequency of the first oscillator and the initial value; and
determining, by the inspection device, whether the internal space contained the first substance, based on the difference.
